(19) United States
(12) Patent Application Publication
(10) Pub. No.: US 2003/0153575 A1
(43) Pub. Date: Aug. 14, 2003

(54) Tetracyclic diketopiperazine compounds as pdev inhibitors

(76) Inventors: Mark W. Orme, Seattle, WA (US); Alain Claude-Marie Daugan, Courtaboeuf (FR); Agnes Bombrun, Monnetier Mornex (FR)

Correspondence Address: MARSHALL, GERSTEIN & BORUN, 6300 SEARS TOWER, 233 SOUTH WACKER, CHICAGO, IL, 60606-6357, US

(21) Appl. No.: 10/296,099

(22) PCT Filed: May 15, 2001
(86) PCT No.: PCT/US01/15937

(51) Int. Cl. A61K 31/498; C07D 487/14

(52) U.S. Cl. 514/249; 544/343

(57) ABSTRACT

Compounds of a general structural formula (I) and salts and solvates thereof, and use of the compounds as PDES inhibitor.

### FIELD AND BACKGROUND OF THE INVENTION

This invention relates to a series of compounds, to methods of preparing the compounds, to pharmaceutical compositions containing the compounds, to their use as therapeutic agents, and to articles of manufacture disclosing the compounds. In particular, the invention relates to compounds that are potent and selective inhibitors of cyclic guanosine 3′,5′-monophosphate specific phosphodiesterase (cGMP-specific PDE), in particular PDE5, and have utility in a variety of therapeutic areas wherein such inhibition is considered beneficial, including the treatment of cardiovascular disorders and erectile dysfunction.

### SUMMARY OF THE INVENTION

The present invention provides compounds of formula (I)

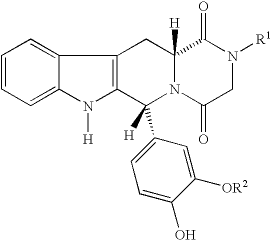

wherein R¹ is C₁₋₆alkyl; and

R² is hydrogen or methyl,

and pharmaceutically acceptable salts and solvates (e.g., hydrates) thereof.

The present invention also provides an article of manufacture for human pharmaceutical use comprising a package insert, a container, and a dosage form of a compound of having a formula

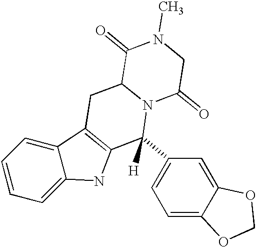

wherein the package insert discloses a compound of formula (Ia)

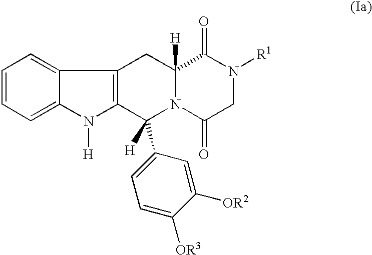

wherein R¹ is C₁₋₆alkyl;

R² is hydrogen or methyl; and

R³ is hydrogen or

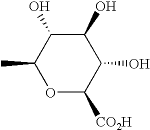

and pharmaceutically acceptable salts ans solvates thereof.

As used herein, the term “alkyl” includes straight chained and branched hydrocarbon groups containing the indicated number of carbon atoms, typically methyl, ethyl, and straight chain and branched propyl and butyl groups. The term “alkyl” includes “cycloalkyl,” which is defined as cyclopropyl, cyclobutyl, cyclohexyl, and cyclopentyl.

The term “container” means any receptacle and closure therefor suitable for storing, shipping, dispensing, and/or handling a pharmaceutical product.

The term “package insert” means information accompanying the product that provides a description of how to administer the product, along with the safety and efficacy data required to allow the physician, pharmacist, and patient to make an informed decision regarding use of the product. The package insert generally is regarded as the “label” for a pharmaceutical product.

Specific compounds of the present invention have the following structures (II) and (III):

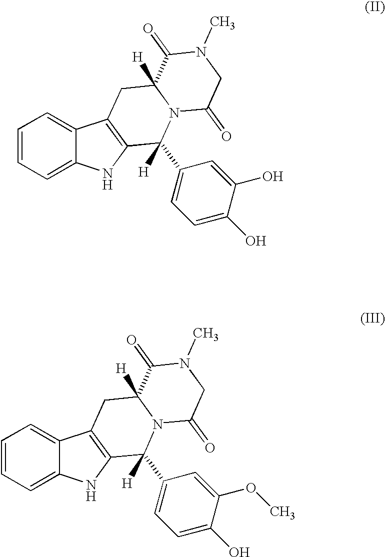

Compounds of formula (I) contain one or more asymmetric center, and, therefore, can exist as stereoisomers. The present invention includes both mixtures and separate individual stereoisomers of the compounds of formula (I). Compounds of formula (I) also can exist in tautomeric forms, and the invention includes both mixtures and separate individual tautomers thereof.

Pharmaceutically acceptable salts of the compounds of formula (I) can be acid addition salts formed with pharmaceutically acceptable acids. Examples of suitable salts include, but are not limited to, the hydrochloride, hydrobromide, sulfate, bisulfate, phosphate, hydrogen phosphate, acetate, benzoate, succinate, fumarate, maleate, lactate, citrate, tartrate, gluconate, methanesulfonate, benzenesulfonate, and p-toluenesulfonate salts. The compounds of the formula (I) also can provide pharmaceutically acceptable metal salts, in particular alkali metal salts and alkaline earth metal salts, with bases. Examples include the sodium, potassium, magnesium, and calcium salts.

Compounds of the present invention are potent and selective inhibitors of cGMP-specific PDE5. Thus, compounds of formula (I) are of interest for use in therapy, specifically for the treatment of a variety of conditions where selective inhibition of PDE5 is considered to be beneficial.

Phosphodiesterases (PDEs) catalyze the hydrolysis of cyclic nucleotides, such as cyclic adenosine monophosphate (cAMP) and cyclic guanosine monophosphate (cGMP). The PDEs have been classified into at least seven isoenzyme families and are present in many tissues (J. A. Beavo, Physiol. Rev., 75, p. 725 (1995)).

PDE5 inhibition is a particularly attractive target. A potent and selective inhibitor of PDE5 provides vasodilating, relaxing, and diuretic effects, all of which are beneficial in the treatment of various disease states. Research in this area has led to several classes of inhibitors based on the cGMP basic structure (E. Sybertz et al., Expert. Opin. Ther. Pat., 7, p. 631 (1997)).

The biochemical, physiological, and clinical effects of PDE5 inhibitors therefore suggest their utility in a variety of disease states in which modulation of smooth muscle, renal, hemostatic, inflammatory, and/or endocrine function is desirable. The compounds of formula (I), therefore, have utility in the treatment of a number of disorders, including stable, unstable, and variant (Prinzmetal) angina, hypertension, pulmonary hypertension, chronic obstructive pulmonary disease, malignant hypertension, pheochromocytoma, congestive heart failure, acute respiratory distress syndrome, acute and chronic renal failure, atherosclerosis, conditions of reduced blood vessel patency (e.g., postpercutaneous transluminal coronary or carotid angioplasty, or post-bypass surgery graft stenosis), peripheral vascular disease, vascular disorders, such as Raynaud's disease; thrombocythemia, inflammatory diseases, myocardial infarction, stroke, bronchitis, chronic asthma, allergic asthma, allergic rhinitis, glaucoma, osteoporosis, preterm labor, benign prostatic hypertrophy, peptic ulcer, male erectile dysfunction, female sexual dysfunction, and diseases characterized by disorders of gut motility (e.g., irritable bowel syndrome).

An especially important use is the treatment of male erectile dysfunction, which is one form of impotence and is a common medical problem. Impotence can be defined as a lack of power, in the male, to copulate, and can involve an inability to achieve penile erection or ejaculation, or both. The incidence of erectile dysfunction increases with age, with about 50% of men over the age of 40 suffering from some degree of erectile dysfunction.

In addition, a further important use is the treatment of female arousal disorder, also termed female sexual arousal disorder. Female arousal disorders are defined as a recurrent inability to attain or maintain an adequate lubrication/swelling response of sexual excitement until completion of sexual activity. The arousal response consists of vasocongestion in the pelvis, vaginal lubrication, and expansion and swelling of external genitalia.

It is envisioned, therefore, that compounds of formula (I) are useful in the treatment of male erectile dysfunction and female sexual arousal disorder. Thus, the present invention concerns the use of compounds of formula (I), or a pharmaceutically acceptable salt thereof, or a pharmaceutical composition containing either entity, for the manufacture of a medicament for the curative or prophylactic treatment of erectile dysfunction in a male animal and sexual arousal disorder in a female animal, including humans.

The term “treatment” includes preventing, lowering, stopping, or reversing the progression or severity of the condition or symptoms being treated. As such, the term “treatment” includes both medical therapeutic and/or prophylactic administration, as appropriate.

It also is understood that “a compound of formula (I),” or a physiologically acceptable salt or solvate thereof, can be administered as the neat compound, or as a pharmaceutical composition containing either entity.

Although the compounds of the invention are envisioned primarily for the treatment of sexual dysfunction in humans, such as male erectile dysfunction and female sexual arousal disorder, they also can be used for the treatment of other disease states.

A further aspect of the present invention, therefore, is providing a compound of formula (I) for use in the treatment of stable, unstable, and variant (Prinzmetal) angina, hypertension, pulmonary hypertension, chronic obstructive pulmonary disease, congestive heart failure, acute respiratory distress syndrome, acute and chronic renal failure, atherosclerosis, conditions of reduced blood vessel patency (e.g., post-PTCA or post-bypass graft stenosis), peripheral vascular disease, vascular disorders such as Raynaud's disease, thrombocythemia, inflammatory diseases, prophylaxis of myocardial infarction, prophylaxis of stroke, stroke, bronchitis, chronic asthma, allergic asthma, allergic rhinitis, glaucoma, osteoporosis, preterm labor, benign prostatic hypertrophy, male and female erectile dysfunction, or diseases characterized by disorders of gut motility (e.g., IBS).

According to another aspect of the present invention, there is provided the use of a compound of formula (I) for the manufacture of a medicament for the treatment of the above-noted conditions and disorders.

In a further aspect, the present invention provides a method of treating the above-noted conditions and disorders in a human or nonhuman animal body which comprises administering to said body a therapeutically effective amount of a compound of formula (I).

Compounds of the invention can be administered by any suitable route, for example by oral, buccal, inhalation, sublingual, rectal, vaginal, transurethral, nasal, topical, percutaneous, i.e., transdermal, or parenteral (including intravenous, intramuscular, subcutaneous, and intracoronary) administration. Parenteral administration can be accomplished using a needle and syringe, or using a high pressure technique, like POWDERJECT™.

Oral administration of a compound of the invention is the preferred route. Oral administration is the most convenient and avoids the disadvantages associated with other routes of administration. For patients suffering from a swallowing disorder or from impairment of drug absorption after oral administration, the drug can be administered parenterally, e.g., sublingually or buccally.

Compounds and pharmaceutical compositions suitable for use in the present invention include those wherein the active ingredient is administered in an effective amount to achieve its intended purpose. More specifically, a “therapeutically effective amount” means an amount effective to prevent development of, or to alleviate the existing symptoms of, the subject being treated. Determination of the effective amounts is well within the capability of those skilled in the art, especially in light of the detailed disclosure provided herein.

A “therapeutically effective dose” refers to that amount of the compound that results in achieving the desired effect. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. Compounds which exhibit high therapeutic indices are preferred. The data obtained from such data can be used in formulating a dosage range for use in humans. The dosage of such compounds preferably lies within a range of circulating concentrations that include the ED₅₀ with little or no toxicity. The dosage can vary within this range depending upon the dosage form employed, and the route of administration utilized.

The exact formulation, route of administration, and dosage can be chosen by the individual physician in view of the patient's condition. Dosage amount and interval can be adjusted individually to provide plasma levels of the active moiety which are sufficient to maintain the therapeutic effects.

The amount of composition administered is dependent on the subject being treated, the subject's weight, the severity of the affliction, the manner of administration, and the judgment of the prescribing physician.

Specifically, for administration to a human in the curative or prophylactic treatment of the conditions and disorders identified above, oral dosages of a compound of formula (I) generally are about 0.5 to about 1000 mg daily for an average adult patient (70 kg). Thus, for a typical adult patient, individual tablets or capsules contain 0.2 to 500 mg of active compound, in a suitable pharmaceutically acceptable vehicle or carrier, for administration in single or multiple doses, once or several times per day. Dosages for intravenous, buccal, or sublingual administration typically are 0.1 to 500 mg per single dose as required. In practice, the physician determines the actual dosing regimen which is most suitable for an individual patient, and the dosage varies with the age, weight, and response of the particular patient. The above dosages are exemplary of the average case, but there can be individual instances wherein higher or lower dosages are merited, and such are within the scope of this invention.

For human use, a compound of the formula (I) can be administered alone, but generally is administered in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of compounds of formula (I) into preparations that can be used pharmaceutically.

These pharmaceutical compositions can be manufactured in a conventional manner, e.g., by conventional mixing, dissolving, granulating, dragee-making, levigating, emulsifying, encapsulating, entrapping, or lyophilizing processes. Proper formulation is dependent upon the route of administration chosen. When a therapeutically effective amount of a compound of the present invention is administered orally, the composition typically is in the form of a tablet, capsule, powder, solution, or elixir. When administered in tablet form, the composition can additionally contain a solid carrier, such as a gelatin or an adjuvant. The tablets capsule, and powder contain about 5% to about 95% compound of the present invention, and preferably from about 25% to about 90% compound of the present invention. When administered in liquid form, a liquid carrier such as water, petroleum, or oils of animal or plant origin can be added. The liquid form of the composition can further contain physiological saline solution, dextrose or other saccharide solutions, or glycols. When administered in liquid form, the composition contains about 0.5% to about 90% by weight of a compound of the present invention, and preferably about 1% to about 50% of a compound of the present invention.

When a therapeutically effective amount of a compound of the present invention is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound of the present invention, an isotonic vehicle.

For oral administration, the compounds can be formulated readily by combining a compound of formula (I) with pharmaceutically acceptable carriers well known in the art. Such carriers enable the present compounds to be formulated as tablets, pills, dragees, capsules, liquids, gels, syrups, slurries, suspensions and the like, for oral ingestion by a patient to be treated. Pharmaceutical preparations for oral use can be obtained by adding a compound of formula (I) with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added.

For administration by inhalation, compounds of the present invention are conveniently delivered in the form of an aerosol spray presentation from pressurized packs or a nebulizer, with the use of a suitable propellant. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the present invention also can be formulated in rectal compositions, such as suppositories or retention enemas, e.g., containing conventional suppository bases. In addition to the formulations described previously, the compounds also can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Many of the compounds of the present invention can be provided as salts with pharmaceutically compatible counterions. Such pharmaceutically acceptable base addition salts are those salts that retain the biological effectiveness and properties of the free acids, and that are obtained by reaction with suitable inorganic or organic bases.

In particular, a compound of formula (I) can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A compound also can be injected parenterally, for example, intravenously, intramuscularly, subcutaneously, or intracoronarily. For parenteral administration, the compound is best used in the form of a sterile aqueous solution which can contain other substances, for example, salts, or monosaccharides, such as mannitol or glucose, to make the solution isotonic with blood.

For veterinary use, a compound of formula (I) or a nontoxic salt thereof, is administered as a suitably acceptable formulation-in accordance with normal veterinary practice. The veterinarian can readily determine the dosing regimen and route of administration that is most appropriate for a particular animal.

Thus, the invention provides in a further aspect a pharmaceutical composition comprising a compound of the formula (I), together with a pharmaceutically acceptable diluent or carrier therefor. There is further provided by the present invention a process of preparing a pharmaceutical composition comprising a compound of formula (I), which process comprises mixing a compound of formula (I), together with a pharmaceutically acceptable diluent or carrier therefor.

In a particular embodiment, the invention includes a pharmaceutical composition for the curative or prophylactic treatment of erectile dysfunction in a male animal, or sexual arousal disorder in a female animal, including humans, comprising a compound of formula (I) or a pharmaceutically acceptable salt thereof, together with a pharmaceutically acceptable diluent or carrier.

Another important aspect of the present invention is to provide an article of manufacture for human pharmaceutical use comprising:

(1) a compound of having a formula

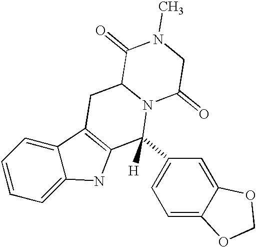

(2) a container, and

(3) a package insert disclosing a compound of formula (Ia)

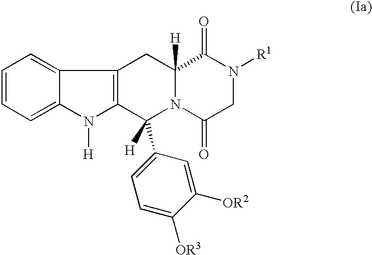

wherein R¹ is C₁₋₆alkyl;

R² is hydrogen or methyl; and

R³ is hydrogen or

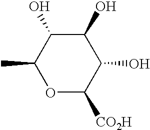

and pharmaceutically acceptable salts and solvates thereof.

The package insert typically identifies a compound of formula (Ia) selected from the group consisting of

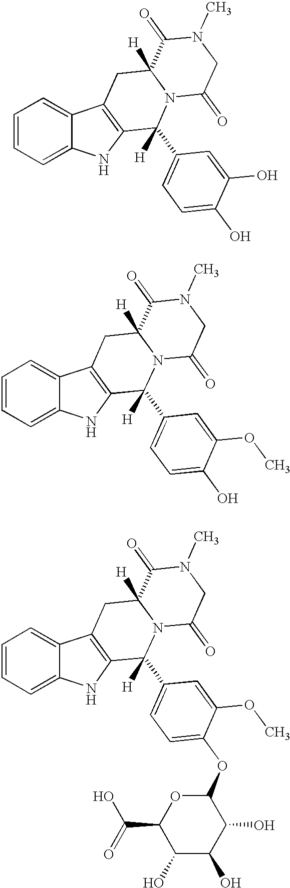

and mixtures thereof.

The package insert provides a description of how to administer a pharmaceutical product, along with the safety and efficacy data required to allow the physician, pharmacist, and patient to make an informed decision regarding the use of the product. The package insert generally is regarded as the label of the pharmaceutical product. The package insert incorporated into the present article of manufacture indicates that the selective PDE5 inhibitor is useful in the treatment of conditions wherein inhibition of PDE5 is desired. The package insert also provides instructions to administer one or more of the unit dosage forms present in the container, as needed.

Preferred conditions to be treated, as set forth in the insert, include sexual dysfunction (including male erectile dysfunction); and female sexual dysfunction, and more preferably female arousal disorder (FAD). The preferred condition to be treated is male erectile dysfunction.

If the article of manufacture contains an oral dosage form, such forms are recognized by those skilled in the art to include, for example, such forms as liquid formulations, tablets, capsules, and gelcaps. Preferably, the dosage forms are solid dosage forms. Any pharmaceutically acceptable excipients for oral use are suitable for preparation of such dosage forms.

The container used in the present article of manufacture is conventional in the pharmaceutical arts. Generally, the container is a blister pack, foil packet, glass or plastic bottle and accompanying cap or closure, or other such article suitable for use by the patient or pharmacist. Preferably, the container is sized to accommodate 1-1000 solid dosage forms, preferably 1 to 500 solid dosage forms, and most preferably, 5 to 30 solid dosage forms.

The compound present in the article of manufacture is a selective PDE5 inhibitor, i.e., (6R-trans)-6-(1,3-benzodioxol-5-yl)-2,3,6,7,12,12a-hexahydro-2-methylpyrazino[1′,2′:1,6]pyrido[3,4-b]indole-1,4-dione, alternatively named (6R,12aR)-2,3,6,7,12,12a-hexahydro-2-methyl-6-(3,4-methylene-dioxyphenyl)pyrazino[2′,1′:6,1]hpyrido[3,4-b]indole-1,4-dione, as disclosed in Daugan U.S. Pat. No. 5,859,006, and represented by the structural formula

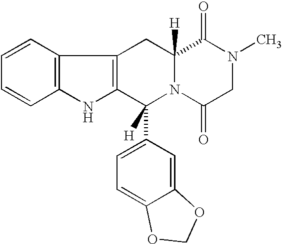

This selective inhibitor can be formulated into tablets, as set forth in U.S. Pat. No. 5,859,006, incorporated herein by reference. The tablets are filled into blister packs and accompanied by a package insert to provide one embodiment of an article of manufacture of the present invention.

Compounds of formula (I) can be prepared by any suitable method known in the art, or by the following processes which form part of the present invention. In the methods below, R¹, R², and R³ are as defined in structural formula (I) above. In particular, Daugan U.S. Pat. No. 5,859,006, incorporated herein by reference, discloses preparation of a compound of structural formula (IV).

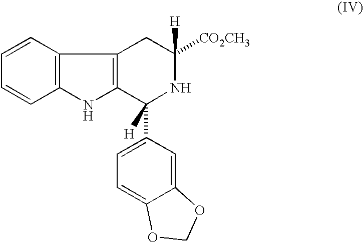

A compound of structural formula (I) is prepared similarly by reacting a tryptophan ester with a suitable aldehyde to provide a desired intermediate. The resulting intermediate then is cyclized by reaction with a suitable amine to provide a compound of structural formula (I). The cyclization reaction is disclosed in Daugan U.S. Pat. No. 5,859,006.

In the synthesis of compounds of structural formula (I), protecting compounds and protecting groups, like benzyl chloroformate and trichloroethyl chloroformate, which are well known to persons skilled in the art, can be used. Such protecting groups are disclosed, for example, in T. W. Greene et al. “Protective Groups in Organic Synthesis, Third Edition,” John Wiley and Sons, Inc., NY, N.Y. (1999).

Compounds of formula (I) an be converted to other compounds of formula (I). Thus, for example, a compound of structural formula (II) can be converted to a compound of structural formula (III).

Compounds of formula (I) can be prepared by the method above as individual stereoisomers from the appropriate stereoisomer of formula (IV) or as a racemic mixture from the appropriate racemic compound of formula (IV). Individual stereoisomers of the compounds of the invention can be prepared from racemates by resolution using methods known in the art for the separation of racemic mixtures into their constituent stereoisomers, for example, using HPLC on a chiral column, such as Hypersil naphthyl urea, or using separation of salts of stereoisomers. Compounds of the invention can be isolated in association with solvent molecules by crystallization from, or evaporation of, an appropriate solvent.

The pharmaceutically acceptable acid addition salts of the compounds of formula (I) that contain a basic center can be prepared in a conventional manner. For example, a solution of the free base can be treated with a suitable acid, either neat or in a suitable solution, and the resulting salt isolated either by filtration or by evaporation under vacuum of the reaction solvent. Pharmaceutically acceptable base addition salts can be obtained in an analogous manner by treating a solution of a compound of formula (I) with a suitable base. Both types of salt can be formed or interconverted using ion-exchange resin techniques. Thus, according to a further aspect of the invention, a method for preparing a compound of formula (I) or a salt or solvate (e.g., hydrate) is provided, followed by (i) salt formation, or (ii) solvate (e.g., hydrate) formation.

The following abbreviations are used hereafter in the accompanying examples: rt (room temperature), min (minute), h (hour), g (gram), mmol (millimole), m.p. (melting point), eq (equivalents), L (liter), mL (milliliter), μL (microliters), DMSO (dimethyl sulfoxide), CH₂Cl₂ (dichloromethane), CHCl₃ (chloroform), CH₃NH₂ (methylamine), IPA (isopropyl alcohol), TFA (trifluoroacetic acid), EtOH (ethanol), MeOH (methanol), Et₃N (triethylamine), AcCl (acetyl chloride), AcOH (acetic acid), DMF (dimethylformamide), EtOAc (ethyl acetate), and THF (tetrahydrofuran).

General Synthesis of a Compound of Formula (I)

The following sequence illustrates a general route to compounds of structural formula (I). One general synthetic route is analogous to the route disclosed in U.S. Pat. No. 5,859,006, incorporated herein by reference. In particular, a tryptophan ester (V) is subjected to a Pictet-Spengler reaction with an appropriate aldehyde, such as (VI) for example, to provide a β-carboline (VII). Acylation of compound (VII) with chloroacetyl chloride provides an N-derivatized compound (VIII), which in turn is treated with a desired primary amine (RNH₂) to provide a diketopiperazine compound (IX) of structural formula (I).

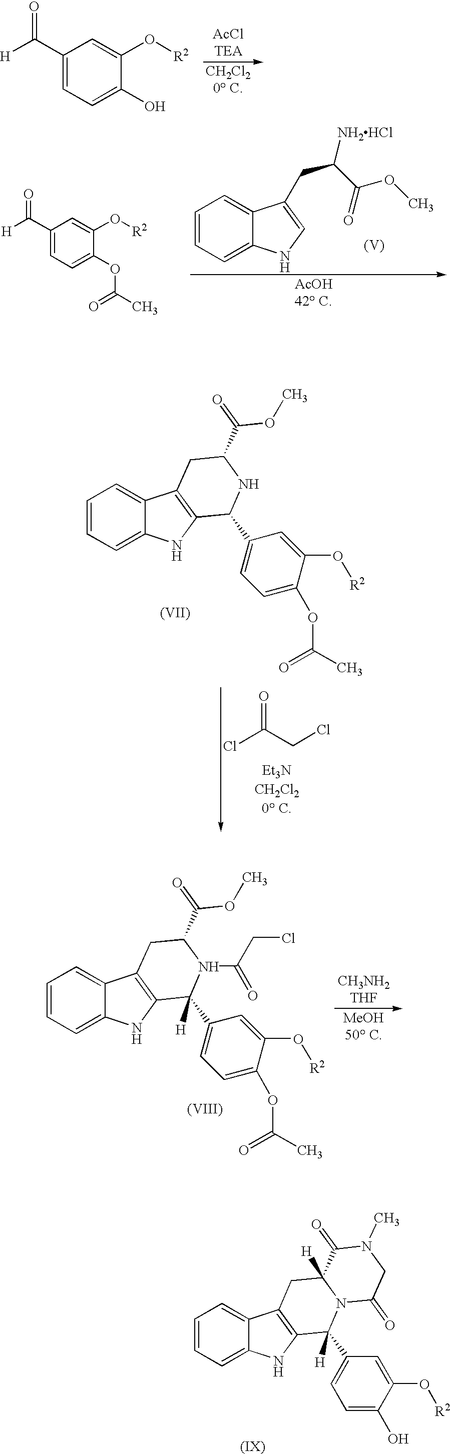

The following illustrates specific examples of compounds of structural formula (I) and synthetic routes to some of these structures.

### PREPARATION OF EXAMPLES 1 AND 2

The following Examples 1 and 2 were prepared by the above-described general synthetic scheme. The details of each step in the above synthetic scheme are disclosed in U.S. Pat. No. 5,849,006.

### PREPARATION OF EXAMPLE 1

### Compound (II)

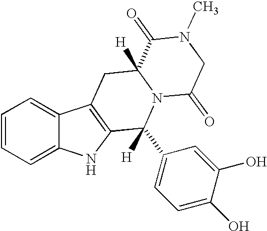

Example 1 was prepared from a tryptophan ester of structural formula (V) and 3,4-dihydroxybenzaldehyde. The tryptophan ester and 3,4-dihydroxybenzaldehyde are available commercially from Aldrich Chemical Co., Milwaukee, Wis.

Preparation of 3,4-Dioxyacetylbenzaldehyde

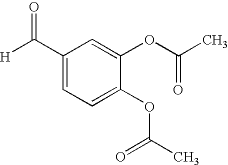

To a mixture of 3,4-dihydroxybenzaldehyde (27.6 g, 0.20 mol) and triethylamine (72 mL, 0.26 mol) in methylene chloride (500 mL), at 0° C., was added acetyl chloride (37 mL, 0.26 mol) dropwise. The reaction mixture (a pink slurry) was stirred at 0° C. for 1 hour, then filtered. The filter cake was washed with methylene chloride (2×20 mL). The combined filtrate next was washed successively with water (100 mL), a mixture of brine (30 mL) and saturated sodium bicarbonate (30 mL), and brine (100 mL), then dried over sodium sulfate. The suspension was clarified by filtration. Concentration of the suspension under reduced pressure yielded a dark brown oil, which was passed through a pad of silica gel. The pad was washed with a 1:1 hexanes:ethyl acetate mixture. The filtrate was concentrated under reduced pressure to yield 97% of the desired product (43.0 g) as an orange oil. R_{f}=0.78 (1:1 ethyl acetate:hexanes). 1H NMR (300 MHz, CDCl₃): δ 9.96 (s, 1H), 7.78-7.80 (m, 1H), 7.74 (d, J=1.8 Hz, 1H), 7.39 (d, J=8.60 Hz, 1H), 2.33 (s, 6H) ppm.

Preparation of cis-1-(3-methoxy-4-hydroxyphenyl)-3-carbomethoxy-1,2,3,4,tetrahydro-β-carboline

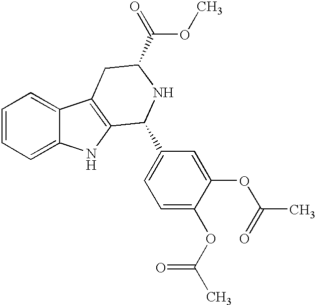

A mixture of D-tryptophan methyl ester hydrochloride (DTME) (24.1 g, 0.095 mol), 3,4-dioxyacetylphenyl (42 g, 0.189 mol), and acetic acid (473 mL) was stirred for 23 hours at 41° C. A second charge of the DTME (15 g, 0.059 mol) then was added to the reaction mixture, and the resulting mixture was stirred at 41° C. for another 19 hours. The resulting solution was analyzed by HPLC (5.3% DTME, 2.8% aldehyde, 59.8% major product, 24.5% minor product). The reaction mixture was diluted with methylene chloride (200 mL), then neutralized with cold saturated aqueous sodium carbonate solution (about 1 L) to pH 7. The aqueous layer was extracted with methylene chloride (200 mL), and the combined organic layer was washed with saturated brine (100 mL), then dried over sodium sulfate. The suspension was clarified by filtration, and the filtrate concentrated under reduced pressure to yield a foam-like yellow solid. The two major products were isolated by column chromatography (8:1 methylene chloride:ethyl acetate to 3:1 methylene chloride:ethyl acetate). The major product (R_{f}=0.50, 3:1 methylene chloride:ethyl acetate) was obtained in 48% yield (38 g) as an off-white solid which was confirmed as the desired cis isomer by NOE analysis (10% enhancement). ¹H NMR (300 MHz, CDCl₃): δ 7.78 (s, 1H), 7.51-7.54 (m, 1H), 7.07-7.19 (m, 6H), 5.19 (s, 1H), 3.93 (dd, J=11.1, 4.0 Hz, 1H), 3.79 (s, 3H), 3.03-3.17 (m, 1H), 2.92-2.99 (m, 1H), 2.28 (s, 3H), 2.24 (s, 3H); ¹³C NMR (75 MHz, CDCl₃: δ 20.4, 20.6, 52.2, 56.8, 57.8, 60.3, 108.8, 111.1, 118.1, 119.6, 122.0, 123.7, 123.8, 126.7, 126.9, 133.8, 136.2, 139.6, 142.1, 142.3, 168.1, 172.9 ppm. The minor product (R_{f}=0.30, 3:1 methylene chloride:ethyl acetate) was obtained in 14% yield (11.2 g) as a pale yellow solid which was confirmed to be the trans isomer by NOE analysis (no enhancement). ¹H NMR (300 MHz, CDCl₃): δ 7.74 (s, 1H), 7.50-7.58 (m, 1H), 7.02-7.30 (m, 6H), 5.35 (s, 1H), 3.93 (m, 1H), 3.72 (s, 3H), 3.04-3.33 (m, 2H), 2.31 (s, 3H), 2.26 (s, 3H) ppm.

Preparation of cis-1-(3,4-dioxyacetylphenyl)-2-chloroacetyl-3-carbomethoxy-1,2,3,4-tetrahydro-β-carboline

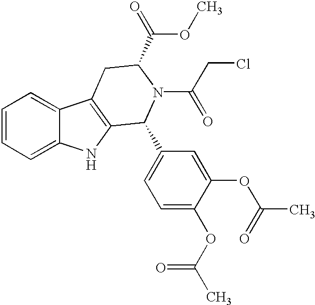

To a mixture of the above cis carboline (34 g, 0.081 mol) and triethylamine (14.5 mL, 0.105 mol) in methylene chloride (255 mL) at 0° C. was added chloroacetyl chloride (8.3 mL, 0.105 mol) dropwise. The mixture was stirred at 0° C. for 1 hour. The resulting yellow solution was washed successively with water (100 mL), a mixture of saturated aqueous sodium bicarbonate solution (50 mL) and brine (50 mL), then dried over sodium sulfate. The suspension was clarified by filtration. The filtrate was concentrated under reduced pressure to provide a 106% yield of the desired product (43 g) as a foam-like yellow solid, which was used without purification. R_{f}=0.73 (3:1 methylene chloride-ethyl acetate).

### Preparation Of Example 1

### Compound II

(6R,12aR)-6-(3,4-Dihydroxyphenyl)-2-methyl-2,3,6,7,12,12a-hexahydropyrazino[1′,2′:1,6]-pyrido[3,4-b]indole-1,4-dione

A mixture of the above chloride (25 g, 0.05 mol), methylamine (150 mL, 2.0 M in THF, 0.30 mol), and methanol (250 mL) was heated at 50° C. for 5 hours. The resulting solution was cooled to ambient temperature to provide a slurry. Filtration under reduced pressure, followed by washing the filter cake with methanol (2×20 mL) yielded a white solid. The white solid was slurried in 0.5 N HCl (400 mL) to remove residual methylamine, then filtered. The filter cake was washed successively with water (300 mL) and hexanes (3×20 mL), then dried in a vacuum oven for 16 hours to yield a 69% yield of Example 1 (13 g) as an off-white solid. m.p. 250.5-256.6° C. R_{f}=0.69 (3:1:0.5 methylene chloride:ethyl acetatemethanol). ¹H-NMR (300 MHz, DMSO-d₆): δ 11.03 (s, 11H), 8.76 (s, 1H), 7.55 (d, J=7.4 Hz, 1H), 7.28 (d, J=7.8 Hz, 1H), 6.96-7.07 (m, 2H), 6.67 (s, 1H); 6.62 (s, 2H), 6.09 (s, 1H), 4.37 (dd, J=17 Hz, 1H), 4.18 (d, J=17 Hz, 1H), 3.91 (d, J=17 Hz, 1H), 3.51 (dd, J=15.9, 4.5, 1H), 2.87-2.99 (m, 4H); ¹³C NMR (75 MHz, DMSO-d₆): δ 22.8, 32.9, 51.6, 54.7, 55.4, 104.4, 111.2, 113.9, 115.1, 117.7, 118.0, 118.8, 121.0, 125.8, 133.7, 134.6, 136.1, 144.3, 144.8, 166.8, 166.8 (the two preceding signals overlap) ppm. Mass spectrum: m/z 378 MH⁺, [α]_{D}^{24°°C.}+83.0 (c=1.0 DMSO). Elemental analysis calculated for C₂₁H₁₉N₃O₄: C, 66.83, H, 5.07, N, 11.13; Found C, 66.44, H, 4.85, N, 10.97. The cis stereo-chemistry was also confirmed by NOE experiment (1.2% enhancement).

### PREPARATION OF EXAMPLE 2

### Compound III

Example 2 was prepared similarly to Example 1, as set forth below.

Preparation of Vanillin Acetate

To a mixture of vanillin (30.4 g, 0.20 mol) and triethylamine (36 ml, 0.26 mol) in methylene chloride (500 mL), at 0° C., was added acetyl chloride (18.5 mL, 0.26 mol) dropwise. The reaction mixture (a white slurry) was stirred at 0° C. for 10 minutes, then filtered. The filter cake was washed with methylene chloride (2×20 mL). The combined filtrate next was washed successively with water (100 mL) and brine (100 mL), then dried over sodium sulfate (Na₂SO₄). The suspension was clarified by filtration, and the filtrate was concentrated under reduced pressure to yield 100% of vanillin acetate (39 g) as a yellow solid. R_{f}=0.5 (3:1, hexanes:ethyl acetate). ¹H NMR (300 MHz, CDCl₃): δ 9.95 (s, 1H), 7.45-7.53 (m, 2H), 7.21-7.25 (m, 1H), 3.90 (s, 3H), 2.37 (s, 3H) ppm.

Preparation of cis-1-(3-methoxy-4-hydroxyphenyl)-3-carbomethoxy-1,2,3,4,tetrahydro-β-carboline

A mixture of D-tryptophan methyl ester hydrochloride (DTME) (25.4 g, 0.10 mol), vanillin acetate (38.8 g, 0.20 mol), and acetic acid (500 mL) was stirred for 17 hours at 42° C. A second charge of DTME (21 g, 0.083 mol) then was added to the amber solution, and the mixture was stirred at 36° C. for another 66 hours. The resulting amber solution was analyzed by HPLC (1.0% DTME, 3.9% aldehyde, 64.8% major product, 25.3% minor product). The reaction mixture was diluted with methylene chloride (400 mL), then neutralized with saturated aqueous sodium carbonate solution to pH 7. The aqueous layer was extracted with methylene chloride (250 mL). The combined organic layer was washed with saturated aqueous sodium chloride (150 mL), then dried over magnesium sulfate. The suspension was clarified by filtration, and the filtrate was concentrated under reduced pressure to yield a foam-like yellow solid (90 g). The two major products were isolated by column chromatography (8:1 methylene chloride:ethyl acetate to 3:1 methylene chloride:ethyl acetate). The major product (R_{f}=0.50, 3:1 methylene chloride:ethyl acetate) was obtained in 58% yield (46 g) as a light yellow solid which was confirmed as the desired cis isomer by NOE analysis (10% enhancement). ¹H NMR (300 MHz, CDCl₃): δ 7.69 (s, 1H), 7.53-7.56 (m, 1H), 7.11-7.26 (m, 3H), 6.93-7.05 (m, 3H), 5.21 (s, 1H), 4.09-4.17 (m, 1H), 3.82 (s, 3H), 3.75 (s, 3H), 3.20-3.27 (m, 1H), 2.93-3.06 (m, 1H), 2.32 (s, 3H). The minor product (r_{f}=0.33, 3:1 methylene chloride:ethyl acetate) was obtained in 15% yield (12 g) as a light yellow solid, and was confirmed to be the trans isomer by NOE analysis (no enhancement). ¹H NMR (300 MHz, CDCl₃): δ 8.13 (s, 1H), 7.54-7.57 (m, 1H), 7.11-7.26 (m, 3H), 6.96 (d, J=8.0 Hz, 1H), 6.88 (s, 1H), 6.75-6.78 (m, 1H), 5.27 (s, 1H), 3.96 (t, J=5.6 Hz, 1H), 3.72 (s, 3H), 3.70 (s, 3H), 3.08-3.30 (m, 2H), 2.30 (s, 3H) ppm.

Preparation of cis-1-(3-methoxy-4-hydroxyphenyl)-2-chloroacetyl-3-carbomethoxy-1,2,3,4,tetrahydro-β-carboline

To a mixture of the above cis carboline (37.5 g, 0.095 mol) and triethylamine (17.2 mL, 0.124 mol) in methylene chloride (300 mL), at 0° C., was added chloroacetyl chloride (9.86 mL, 0.124 mol) dropwise. The mixture was stirred at 0° C. for 0.5 hours. The resulting yellow solution was washed successively with water (100 mL), saturated aqueous sodium bicarbonate solution (50 mL) and saturated brine (100 mL), then dried over sodium sulfite. The suspension was clarified by filtration and the filtrate was concentrated under reduced pressure to provide a 112% crude yield (50 g) of the desired product as a foam-like yellow solid, which was used without purification. R_{f}=0.72 (3:1 methylene chloride:ethyl acetate).

### PREPARATION OF EXAMPLE 2

### Compound III

(6R,12aR)-6-(4-Hydroxy-3-methoxyphenyl)-2-methyl-2,3,6,7,12,12a-hexahydropyrazino[1′,2′:1,6]-pyrido[3,4-b]indole-1,4-dione

A mixture of the crude chloride (24 g, 0.051 mol), methylamine (127 mL, 2.0 M in THF, 0.274 mol), and methanol (189 mL) was heated at 50° C. for 5 hours. The resulting suspension was cooled to ambient temperature. Filtration under reduced pressure, followed by washing with methanol (2×20 mL) provided an 89% yield of Example 2 (13 g) as an off-white solid. m.p. 288.2-292.2° C., R_{f}=0-0.49 (3:1:0.2 methylene chloride:ethyl acetate:methanol). ¹H NMR (300 MHz, DMSO-d₆): δ 11.01 (s, 1H), 8.85 (S, 1H), 7.53 (d, J=7.5 Hz, 1H), 7.29 (d, J=7.5 Hz, 1H), 6.95-7.07 (m, 3H), 6.61 (S, 2H), 6.15 (S, 1H), 4.39 (dd, J=11.6, 4.3 Hz, 1H), 4.18 (d, J=17.0 Hz, 1H), 3.92 (d, J=17.0 Hz, 1H), 3.72 (S, 3H), 3.50 (dd, J=15.7, 4.5 Hz, 1H), 3.36 (S, 3H), 2.90-2.99 (m, 4H); ¹³C NMR (75 MHz DMSO-d₆): δ 22.86, 32.88, 51.55, 54.90, 55.43, 55, 62, 104.45, 111.17, 111.26, 115.24, 117.99, 118.79, 121.07, 133.84, 134.36, 136.06, 145.51, 147.13, 166.81, 166.81 (the two preceding signals overlap) ppm. Mass spectrum: m/z 392 MH⁺, [α]_{D}^{24° C.}+46.4 (c=1.0, DMSO). Elemental analysis calculated for C₂₂H₂₁N₃O₄:C, 67.51, H, 5.41, N, 10.74; Found C, 67.19, H, 5.51, N, 10.71.

Compounds of the present invention can be formulated into tablets for oral administration. For example, a compound of formula (I) can be formed into a dispersion with a polymeric carrier by the coprecipitation method set forth in WO 96/38131, incorporated herein by reference. The coprecipitated dispersion can be blended with excipients, then pressed into tablets, which optionally are film-coated.

The compounds of structural formula (I) were tested for an ability to inhibit PDE5. The ability of a compound to inhibit PDE5 activity is related to the IC₅₀ value for the compound, i.e., the concentration of inhibitor required for 50% inhibition of enzyme activity. The IC₅₀ value for compounds of structural formula (I) were determined using recombinant human PDE5.

The compounds of the present invention typically exhibit an IC₅₀ value against recombinant human PDE5 of less than about 50 μM, and preferably less than about 25 μM, and more preferably less than about 15 μm. The compounds of the present invention typically exhibit an IC₅₀ value against recombinant human PDES of less than about 1 μM, and often less than about 0.05 μM. To achieve the full advantage of the present invention, a present PDE5 inhibitor has an IC₅₀ of about 0.1 nM to about 15 μM.

The production of recombinant human PDEs and the IC₅₀ determinations can be accomplished by well-known methods in the art. Exemplary methods are described as follows:

Expression of Human PDEs

Expression in Saccharomyces cerevisiae (Yeast)

Recombinant production of human PDE1B, PDE2, PDE4A, PDE4B, PDE4C, PDE4D, PDE5, and PDE7 was carried out similarly to that described in Example 7 of U.S. Pat. No. 5,702,936, incorporated herein by reference, except that the yeast transformation vector employed, which is derived from the basic ADH2 plasmid described in Price et al., Methods in Enzymology, 185, pp. 308-318 (1990), incorporated yeast ADH2 promoter and terminator sequences and the Saccharomyces cerevisiae host was the protease-deficient strain BJ2-54 deposited on Aug. 31, 1998 with the American Type Culture Collection, Manassas, Va., under accession number ATCC 74465. Transformed host cells were grown in 2×SC-leu medium, pH 6.2, with trace metals, and vitamins. After 24 hours, YEP medium-containing glycerol was added to a final concentration of 2×YET/3% glycerol. Approximately 24 hr later, cells were harvested, washed, and stored at −70° C.

Human Phosphodiesterase Preparations

Phosphodiesterase Activity Determinations

Phosphodiesterase activity of the preparations was determined as follows. PDE assays utilizing a charcoal separation technique were performed essentially as described in Loughney et al. (1996). In this assay, PDE activity converts [³²P]cAMP or [³²P] cGMP to the corresponding [³²P]5′-AMP or [³²P]5′-GMP in proportion to the amount of PDE activity present. The [³²P]5′-AMP or [³²P]5′-GMP then was quantitatively converted to free [³²P]phosphate and unlabeled adenosine or guanosine by the action of snake venom 5′-nucleotidase. Hence, the amount of [³²P] phosphate liberated is proportional to enzyme activity. The assay was performed at 30° C. in a 100 μL reaction mixture containing (final concentrations) 40 mM Tris HCl (pH 8.0), 1 μM ZnSO₄, 5 mM MgCl₂, and 0.1 mg/mL bovine serum albumin (BSA). PDE enzyme was present in quantities that yield <30% total hydrolysis of substrate (linear assay conditions). The assay was initiated by addition of substrate (1 mM [³²P]cAMP or cGMP), and the mixture was incubated for 12 minutes. Seventy-five (75) μg of Crotalus atrox venom then was added, and the incubation was continued for 3 minutes (15 minutes total). The reaction was stopped by addition of 200 pL of activated charcoal (25 mg/mL suspension in 0.1 M NaH₂PO₄, pH 4). After centrifugation (750×g for 3 minutes) to sediment the charcoal, a sample of the supernatant was taken for radioactivity determination in a scintillation counter and the PDE activity was calculated.

Purification of PDE5 from S. cerevisiae

Cell pellets (29 g) were thawed on ice with an equal volume of Lysis Buffer (25 mM Tris HCl, pH 8, 5 mM MgCl₂, 0.25 mM DTT, 1 mM benzamidine, and 10 μM ZnSO₄). Cells were lysed in a Microfluidizer (Microfluidics Corp.) using nitrogen at 20,000 psi. The lysate was centrifuged and filtered through 0.45 μm disposable filters. The filtrate was applied to a 150 mL column of Q SEPHAROSE® Fast-Flow (Pharmacia). The column was washed with 1.5 volumes of Buffer A (20 mM Bis-Tris Propane, pH 6.8, 1 mM MgCl₂, 0.25 mM DTT, 10 μM ZnSO₄) and eluted with a step gradient of 125 mM NaCl in Buffer A followed by a linear gradient of 125-1000 mM NaCl in Buffer A. Active fractions from the linear gradient were applied to a 180 mL hydroxyapatite column in Buffer B (20 mM Bis-Tris Propane (pH 6.8), 1 mM MgCl₂, 0.25 mM DTT, 10 μM ZnSO₄, and 250 mM KCl). After loading, the column was washed with 2 volumes of Buffer B and eluted with a linear gradient of 0-125 mM potassium phosphate in Buffer B. Active fractions were pooled, precipitated with 60% ammonium sulfate, and resuspended in Buffer C (20 mM Bis-Tris Propane, pH 6.8, 125 mM NaCl, 0.5 mM DTT, and 10 μM ZnSO₄). The pool was applied to a 140 mL column of SEPHACRYL® S-300 HR and eluted with Buffer C. Active fractions were diluted to 50% glycerol and stored at −20° C.

The resultant preparations were about 85% pure by SDS-PAGE. These preparations had specific activities of about 3 μmol cGMP hydrolyzed per minute per milligram protein.

Inhibitory Effect on cGMP-PDE

cGMP-PDE activity of compounds of the present invention was measured using a one-step assay adapted from Wells et al., Biochim. Biophys. Acta, 0.384, 430 (1975). The reaction medium contained 50 mM Tris-HCl, pH 7.5, 5 mM magnesium acetate, 250 μg/ml 5′-Nucleotidase, 1 mM EGTA, and 0.15 μM 8-[H³]-cGMP. Unless otherwise indicated, the enzyme used was a human recombinant PDE5 (ICOS Corp., Bothell, Wash.).

Compounds of the invention were dissolved in DMSO finally present at 2% in the assay. The incubation time was 30 minutes during which the total substrate conversion did not exceed 30%.

The IC₅₀ values for the compounds examined were determined from concentration-response curves typically using concentrations ranging from 10 nM to 10 μM. Tests against other PDE enzymes using standard methodology showed that compounds of the invention are selective for the cGMP-specific PDE enzyme.

Biological Data

Compounds of the present invention typically were found to exhibit an IC₅₀ value of less than 500 nM, and usually less than 250 nM. In vitro test data for representative compounds of the invention is given in the following table:

TABLE 1
In vitro results

| Example | PDE5 IC₅₀ (nM) |
|---|---|
| 1 | 45 |
| 2 | 230 |

Obviously, many modifications and variations of the invention as hereinbefore set forth can be made without departing from the spirit and scope thereof, and, therefore, only such limitations should be imposed as are indicated by the appended claims.

What is claimed is:

1. A compound having a formula 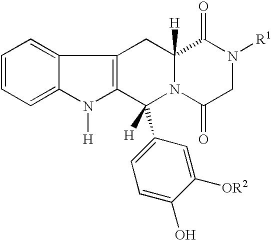
wherein R¹ is C₁₋₆alkyl; and
R² is hydrogen or methyl;
and pharmaceutically acceptable salts and solvates thereof.

2. The compound of claim 1 wherein R² is hydrogen.

3. The compound of claim 1 wherein R² is methyl.

4. The compound of claim 1 having the formula

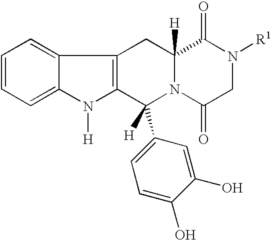

5. The compound of claim 1 selected from the group consisting of 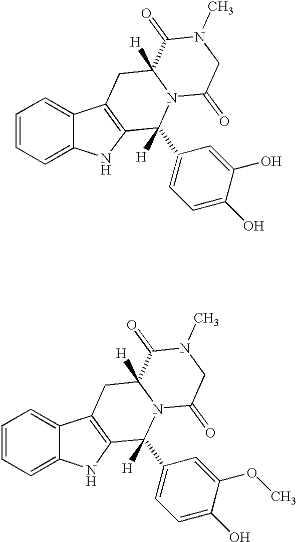
and pharmaceutically acceptable salts and solvates thereof.

6. A pharmaceutical composition comprising a compound of claim 1, together with a pharmaceutically acceptable diluent or carrier.

7. A method of treating a male or female animal in the treatment of a condition where inhibition of a cGMP-specific PDE is of a therapeutic benefit comprising treating said animal with an effective amount of a pharmaceutical composition comprising a compound of claim 1, together with a pharmaceutically acceptable diluent or carrier.

8. The method of claim 7 wherein the condition is male erectile dysfunction.

9. The method of claim 8 wherein the treatment is an oral treatment.

10. The method of claim 7 wherein the condition is female arousal disorder.

11. The method of claim 10 wherein the treatment is an oral treatment.

12. The method of claim 7 wherein the condition is selected from the group consisting of stable angina, unstable angina, variant angina, hypertension, pulmonary hypertension, chronic obstructive pulmonary disease, malignant hypertension, pheochromocytoma, acute respiratory distress syndrome, congestive heart failure, acute renal failure, chronic renal failure, atherosclerosis, a condition of reduced blood vessel patency, a peripheral vascular disease, a vascular disorder, thrombocythemia, an inflammatory disease, myocardial infarction, stroke, bronchitis, chronic asthma, allergic asthma, allergic rhinitis, glaucoma, peptic ulcer, a gut motility disorder, postpercutaneous transluminal coronary angioplasty, carotid angioplasty, post-bypass surgery graft stenosis, osteoporosis, preterm labor, benign prostatic hypertrophy, and irritable bowel syndrome.

13. A method of treating a condition where inhibition of a cGMP-specific PDE is of therapeutic benefit, in a human or a nonhuman animal body, comprising administering to said body a therapeutically effective amount of a compound of claim 1.

14. A method for the curative or prophylactic treatment of male erectile dysfunction or female arousal disorder, comprising administration of an effective dose of a compound of claim 1, and pharmaceutically acceptable salts and solvates thereof, to an animal.

15. Use of a compound of claim 1 for the manufacture of a medicament for the curative or prophylactic treatment of a condition where inhibition of a cGMP-specific PDE is of a therapeutic benefit.

16. An article of manufacture for human pharmaceutical use comprising:
(i) a compound having a formula 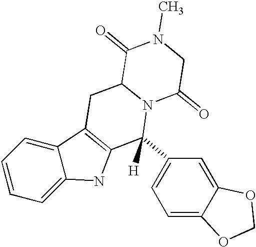
(ii) a container, and
(iii) a package insert disclosing a compound of formula 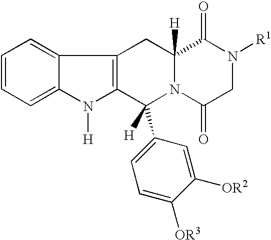
wherein R¹ is C₁₋₆alkyl;
R² is hydrogen or methyl; and
R³ is hydrogen or 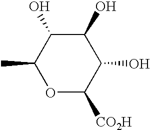
and pharmaceutically acceptable salts and solvates thereof.

17. The article of claim 16 wherein the package insert identifies the compound in (iii) as being a cGMP PDE5 inhibitor.

18. The article of claim 17 wherein the package insert identifies the compound in (iii) as being useful in treating male erectile dysfunction or female sexual arousal disorder.

19. The article of any one of claims 16 through 18 wherein the package insert identifies a compound in (iii) selected from the group consisting of 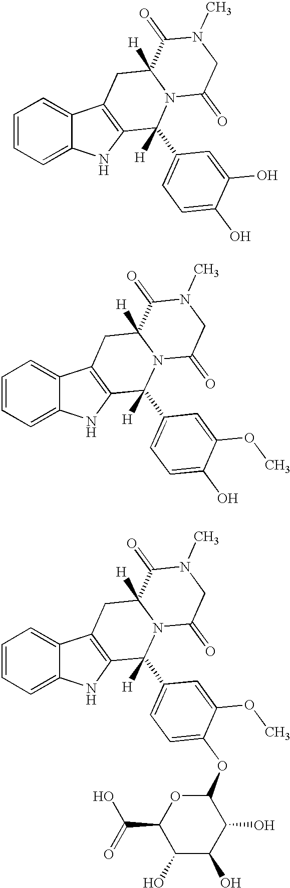
and mixtures thereof.
